(19) United States
(12) Patent Application Publication
(10) Pub. No.: US 2010/0093863 A1
(43) Pub. Date: Apr. 15, 2010

(54) USE OF CITRULLINE FOR PREVENTING AN INCREASE IN PROTEIN CARBONYLATION AND FOR TREATING DISEASES RESULTING THEREFROM

(75) Inventors: Christophe Moinard, Bourg La Reine (FR); Stephane Walrand, Aydat (FR); Yves Boirie, Beaumont (FR); Luc Cynober, Sceaux (FR)

Correspondence Address: YOUNG & THOMPSON, 209 Madison Street, Suite 500, Alexandria, VA, 22314, US

(73) Assignees: UNIVERSITE PARIS DESCARTESURE, Paris Cedex 06 (FR); INSTITUT NATIONAL DE LA RECHERCHE AGRONOMIQUE, Paris Cedex 07 (FR); ASSISTANCE PUBLIQUE-HOPITAUX DE PARIS, Paris Cedex 04 (FR); UNIVERSITE D' AUVERGNE, Clermont-Ferrand (FR)

(21) Appl. No.: 12/532,299

(22) PCT Filed: Mar. 20, 2008
(86) PCT No.: PCT/FR2008/000379

§ 371 Date: Dec. 10, 2009

(30) Foreign Application Priority Data
Mar. 22, 2007 (FR) 0702090

(51) Int. Cl. A61K 31/197; A61P 9/10

(52) U.S. Cl. 514/563

(57) ABSTRACT

A method of using L-citrulline of the formula (I) for preparing a cosmetic composition, a food or nutraceutic composition or a pharmaceutical composition for treating diseases related to an increase in protein carbonylation, in particular for treating neurodegenerative diseases such as Alzheimer's or Parkinson's disease.

A subject of the present invention is the novel use of citrulline, namely its use within the framework of an increase in the carbonylation of proteins and the treatment of pathologies resulting from it.

It has been clearly established that proteins in extra- and intracellular media constitute in vivo a preferred target for reactive oxygen species and reactive nitrogen species. It has been estimated that proteins can capture up to 50 to 70% of radical species synthesized by the cell. Several types of damage are observed: oxidation (or nitration) of the side chains of amino acids, or oxidation of the polypeptide chain (of which carbonylation is a particular case) followed by breakdown and/or formation of inter- or intrachain bonds.

Carbonylation of proteins is a phenomenon which can be independent of the phenomenon of oxidation.

Together these modifications to the proteins can cause modifications in structure a consequence of which can be their deactivation, the latter possibly causing damage to the cell. Moreover, in the current state of knowledge there does not appear to be a system for repairing carbonylated proteins.

The expression “carbonylation of proteins” denotes a chemical modification of proteins linked to the production of CO groups. This carbonylation (which can be a particular case of the oxidation of proteins) then causes a loss in the function of the protein (see Nyström, The EMBO Journal (2005) 24, 1311-1317 and Dalle-Donne et al., J. Cell. Mol. Med., Vol. 10, no. 2, 2006, pp. 389-406). Although the organism has a certain number of defence systems (glutathione, catalase, SOD etc.), in certain situations these can be overcome and prove insufficient.

Carbonylated compounds constitute a useful marker which accompanies aging and numerous pathologies. In fact, these physiopathologic al situations (neurodegenerative diseases, inflammatory diseases, etc.) are characterized by an increase in carbonylated proteins in tissues. It is therefore essential to develop strategies aimed at decreasing the carbonylation of proteins.

Citrulline (or 2-amino-5-(carbamoylamino)pentanoic acid) is an α-amino acid and was first isolated from watermelon. Citrulline is a non-essential amino acid which the organism produces from other nutrients. For example, citrulline plays a particularly important part, with ornithine and arginine, in the urea cycle. Finally, citrulline plays a major part in the homeostasis of arginine and nitric oxide.

Citrulline is currently used within the scope of certain pharmaceutical applications. Thus European patent application EP 1 495 755 relates to the use of citrulline for the preparation of a medicament for the treatment of pathologies linked to intestinal insufficiency. More particularly, the pathologies mentioned in this application are as follows: short-bowel syndrome following an intestinal resection, celiac disease, chronic inflammatory diseases of the intestine, intestinal insufficiency linked to aging and intestinal insufficiency linked to irradiation. However, this application does not mention the carbonylation of proteins.

International application WO 2005/115371 describes pharmaceutical compositions comprising a statin combined with citrulline for the preparation of a medicament used in the treatment of primary or secondary atherosclerosis, and in that of degenerative diseases such as Alzheimer's disease. The use of these compositions has a synergy comparable with compositions comprising a statin or citrulline on its own.

Application FR 2691359 describes the use of citrulline malate in a patient suffering from chronic obstructive pulmonary disease (COPD) and in a patient suffering from Reye-Johnson syndrome (mitochondrial myopathy). This beneficial effect of citrulline malate is assumed to be due to an improved use of fats in the muscles leading to a reduction in muscle proteolysis.

The American patent application describes the oral use of L-citrulline as a food supplement in the case of neurodegenerative diseases (dementias, Alzheimer's disease, Parkinson's disease), retinopathies (age-related macular degeneration) and amyotrophic lateral sclerosis. This citrulline intake increases the plasma concentration of arginine and therefore the availability of the latter for the generation of nitric oxide. In these indications, citrulline is combined with phytate and calcium carbonate.

The American patent application US 2004/0235953 describes and claims a method for the prevention and therapeutic treatment of pathologies associated with a decrease in the formation of nitric oxide, in particular sepsis (systemic infection), said method comprising the administration of a precursor of nitric oxide which can be citrulline.

The American patent application US 2001/0056068 relates to the use of L-citrulline for treating diseases associated with a nitric oxide deficiency such as atherosclerosis, cerebral ischemia and Alzheimer's disease.

XP 00 24 56 361 discloses ophthalmic formulations containing citrulline and their use in Chinese medicine in the treatment of cataracts. These formulations act on the ciliary vascular system and the pericorneal vasoganglion.

The European patent application EP 1752156 describes an oxygen scavenging agent containing an extract of wild watermelon containing citrulline and its use in particular in Alzheimer-type dementia, cataract problems, and cutaneous lesions.

Although these documents describe the use of citrulline on its own or in combination for the preparation of medicaments used in the treatment of neurodegenerative diseases such as Alzheimer's disease, atherosclerosis, cataract problems or systemic infections (sepsis) and in the treatment of mitochondrial myopathies, and nutraceutical compositions suitable for use within the framework of neurodegenerative diseases (dementias, Alzheimer's disease, Parkinson's disease), retinopathies (age-related macular degeneration) and amyotrophic lateral sclerosis, none of these documents describes the direct effect of citrulline on the carbonylation of proteins.

Moreover, one of the aims of the present invention is to provide a means of preventing and treating an increase in the carbonylation of proteins and the pathologies resulting from it.

The present invention relates to the use of L-citrulline of the following formula (I):

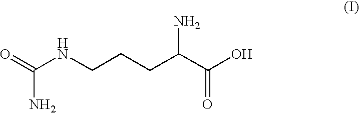

or of a pharmaceutically acceptable salt thereof,
for the preparation of a pharmaceutical composition intended to inhibit an increase in the carbonylation of the relevant proteins observed in certain pathologies and therefore intended in particular for the treatment of neurodegenerative diseases such as Alzheimer's disease or Parkinson's disease, for the treatment of retinopathy, rheumatoid polyarthritis, atherosclerosis, amyotrophic lateral sclerosis, cerebral ischemia, for the treatment of cataract problems or systemic infections (sepsis), for the treatment of pathologies associated with cutaneous aging such as for example wrinkles, hypo- and hyperpigmented spots and loss of elasticity and more particularly with senescence in tissues such as the eye, muscle and brain, for the treatment of pathologies associated with mitochondrial dysfunction, namely myopathies resulting from mitochondriopathies, for the treatment of cachexia associated with malnutrition and with increased longevity in mammals, in particular domestic animals.

A subject of the present invention is also the use of a composition comprising L-citrulline or of a cosmetically acceptable salt thereof, for the cosmetic treatment of disorders associated with the carbonylation of the relevant proteins, in particular in the case of pathologies associated with cutaneous aging such as for example wrinkles, hypo- and hyperpigmented spots and loss of elasticity.

A subject of the present invention is also the use of a composition comprising L-citrulline or of a nutraceutically acceptable salt thereof, for the nutraceutical treatment of disorders associated with an increase in the carbonylation of proteins, by inhibiting an increase in the carbonylation of the relevant proteins, therefore intended in particular for the treatment of neurodegenerative diseases such as Alzheimer's disease or Parkinson's disease, for the treatment of retinopathy, rheumatoid polyarthritis, atherosclerosis, amyotrophic lateral sclerosis, cerebral ischemia, for the treatment of cataract problems or systemic infections (sepsis), for the treatment of pathologies associated with cutaneous aging such as for example wrinkles, hypo- and hyperpigmented spots and loss of elasticity and more particularly with senescence in tissues such as the eye, muscle and brain, for the treatment of pathologies associated with mitochondrial dysfunction, namely myopathies resulting from mitochondriopathies, for the treatment of cachexia associated with malnutrition and with increased longevity in mammals.

By nutraceutical is meant any product making health claims (health food, functional food, borderline products etc.).

A subject of the present invention is also the use of a composition comprising L-citrulline, said composition being intended as a dietary supplement suitable for use within the framework of neurodegenerative diseases such as Alzheimer's disease or Parkinson's disease, retinopathy, rheumatoid polyarthritis, atherosclerosis, amyotrophic lateral sclerosis, cerebral ischemia, cataract problems or systemic infections (sepsis), cutaneous aging such as for example wrinkles, hypo- and hyperpigmented spots and loss of elasticity and more particularly with senescence in tissues such as the eye, muscle and brain, pathologies associated with mitochondrial dysfunction, namely myopathies resulting from mitochondriopathies, for the treatment of cachexia associated with malnutrition and with increased longevity in mammals, in particular domestic animals.

By L-citrulline within the scope of the invention is meant the commercially available product, in particular as supplied by Sigma, Biocodex or Kyowa Hakko or the natural product originating from plants, in particular watermelon (Citrullus lanatus), in particular in juice, pulp or extract form.

By “pharmaceutically acceptable salt”, “cosmetically acceptable salt” and “nutraceutically acceptable salt” is meant citrulline salts such as citrulline malate, citrulline α-ketoglutarate, citrulline citrate or citrulline α-ketoisocaproate.

In an advantageous embodiment of the invention, the L-citrulline as defined above is used for the preparation of a pharmaceutical composition, a composition intended for cosmetic treatment, a composition intended as a dietary supplement, and a composition intended for nutraceutical treatment or as a health food such that the unit L-citrulline dose is about 2 g to about 20 g, in particular about 10 g, for a dosage of about 0.1 g/kg/day to about 0.5 g/kg/day, in particular about 0.25 g/kg/day.

In an advantageous embodiment of the invention, L-citrulline is taken in one to three daily doses, preferably in one dose.

According to another advantageous embodiment of the invention, the pharmaceutical composition and the composition for nutraceutical treatment (or as a health food) are in dried form, in the form of an aqueous solution, in hydroalcoholic or oily form, in the form of an oil-in-water or water-in-oil or multiple emulsion, or of an aqueous or oily gel. Any suitable vehicle i.e. any cosmetically, in particular dermatologically, acceptable excipient, vehicle or support can be used for the composition for cosmetic treatment. Such supports are well-known to a person skilled in the art and are also produced from cosmetic or dermatological compositions which have a general application. For example, mixtures, formulations, monolamellar encapsulations (micelles), bilamellar encapsulations (liposomes), multilamellar encapsulations (spherulites) and any other support known in cosmetics can be used.

The present invention relates to the use of L-citrulline as defined above, for the preparation of a pharmaceutical composition in a form administrable by oral, enteral or parenteral route. In the case of enteral nutrition or parenteral nutrition, it can be mixed with the food or preferably administered as a bolus in an enteral gastric probe or in a Y-tube in parenteral nutrition.

It also relates to the use of L-citrulline as defined above for the preparation of a composition for cosmetic treatment in a form administrable by topical route. It also relates to the use of L-citrulline as defined above for the preparation of a composition for nutraceutical treatment in a form administrable by oral route.

Administration by enteral route corresponds in particular to administration by nasogastric or nasointestinal probe, by gastrostomy or jejunostomy; administration by parenteral route corresponds in particular to administration by central, peripheral or subcutaneous intravenous perfusion.

According to a preferred embodiment, L-citrulline is used within the scope of the invention for the preparation of a pharmaceutical composition, a composition for cosmetic treatment, a composition for a dietary supplement or health food for nutraceutical treatment also comprising one or more other compounds intended for the treatment of cachexia associated with malnutrition, such as leucine, glutamine, arginine, ornithine and their various usable salts such as α-ketoglutarate or α-ketoisocaproate.

Within the framework of a composition for nutraceutical treatment, the compounds can be used on their own or in a nutrient mixture intended for oral nutrition.

According to a preferred embodiment, L-citrulline is used within the scope of the invention for the preparation of a pharmaceutical composition, a composition intended for cosmetic treatment, a dietary supplement or a composition intended for nutraceutical treatment (health food) also comprising one or more other compounds intended for the treatment of cachexia associated with malnutrition such as leucine, glutamine, arginine, ornithine and their various usable salts such as α-ketoglutarate or α-ketoisocaproate, on their own or in a nutrient mixture intended for oral nutrition.

According to another embodiment, the invention relates to products comprising:
L-citrulline or a pharmaceutically or nutraceutically acceptable salt thereof,
and at least one other compound intended for the treatment of cachexia associated with malnutrition, such as leucine, glutamine, arginine, ornithine and their various usable salts such as α-ketoglutarate or α-ketoisocaproate, on their own or in a nutrient mixture intended for parenteral nutrition, or as a mixture intended for enteral nutrition or a mixture intended for oral nutrition, as combination products for simultaneous or separate use, or sequential use within the framework of the treatment of pathologies associated with an increase in the carbonylation of proteins, said pathologies being as defined above.

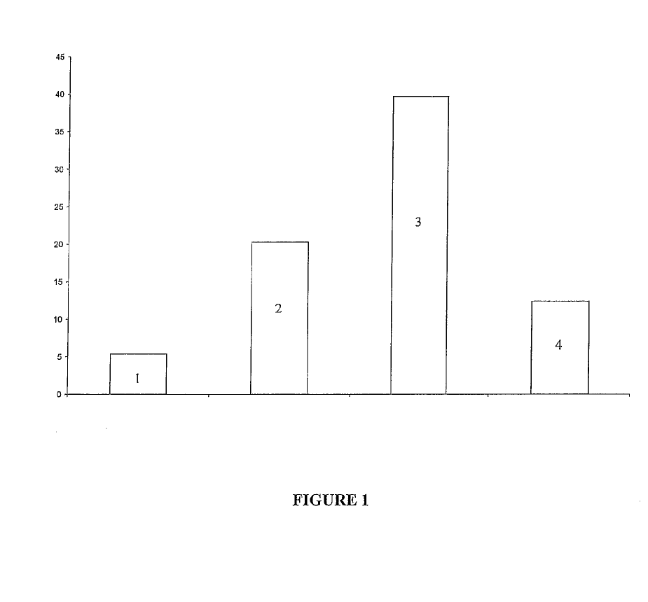

Examples 1 and 2 below and FIG. 1 illustrate the invention.

FIG. 1 represents the carbonylation of muscle proteins in rats. The y-axis corresponds to the quantity of carbonylated proteins in μmol/g. Column “1” corresponds to healthy rats; column “2” corresponds to undernourished rats; column “3” corresponds to rats renourished with a standard diet and column “4” corresponds to rats renourished with a diet enriched with citrulline.

### EXAMPLE 1

### Influence of Citrulline in Malnourished-Renourished Rats

Materials and Methods

I—Materials

All the chemical reagents used come from Sigma (Saint-Quentin-Fallavier, France). The L-citrulline was supplied free of charge by Laboratoires Biocodex (Compiègne, France).

II—Treatment of the Animals

Male Sprague-Dawley rats (Charles River Laboratoires, L'Arbresles, France) aged 19 months are placed in individual cages in a thermostatically-controlled atmosphere (23°±1° C.) and subjected to a 12-h light-darkness cycle (darkness from 8 h to 20 h). The manager of the programme is authorized (no. 75.522) by the Ministry of Agriculture and Forestry to carry out this type of experiment. Moreover, the use and the treatment of the laboratory animals comply with legislation (D2001-486) and are in accordance with European legislation (Official Journal of the European Community L358 Dec. 18, 1986).

The rats are acclimatized for 2 weeks during which spontaneous food consumption is measured. They are fed with a standard diet (A04, UAR, Villemoisson-sur-Orge, France) containing 17% proteins, 3% lipids, 59% glucides and 21% water, fibres, vitamins and minerals. The average food intake during this period is 34.4 g/day.

III—Experimental Protocol

After the acclimatization period, the rats are randomized into 4 groups: A control group made up of rats (n=10) fed ad libitum (AL) for 12 weeks and 3 other groups subjected to a restricted diet during the same period: fed with just 50% of the spontaneous ingesta (or 17.2 g), with the standard diet (UAR A04). At the end of the restriction period, the animals (n=10) of one group are sacrificed (group R) and the rats of the two remaining groups are renourished for one week with 90% of the spontaneous ingesta (or 30.9 g), or with a diet enriched with non-essential amino acids (group AANE: alanine, asparagine, glycine, serine, histidine, and proline provided in equimolar quantities) or a diet enriched with citrulline (5 g/kg/d) (citrulline group). The intakes of the two groups are isonitrogenous and isocaloric. The limitation to 90% of the spontaneous intakes ensures that the rats consume all of the food offered to them.

III.1 Evaluation of the Carbonylation Level of the Proteins

The processes of oxidation of the proteins affecting the structure of the amino acids which comprise them are complex and all give rise to the formation of carbonyl groups. For this reason it was decided to carry out an overall evaluation of the oxidation of the muscle proteins by quantifying the carbonylated derivatives from a sample of 300 mg of tibialis anterior. The method used is therefore a non-specific amino-acid type technique based on the spectrophotometric assay of the complex formed by binding a compound called dinitrophenyl-hydrazine (DNPH) to the carbonyl groups. The muscle proteins are isolated. The protein pellet is taken up in 600 μl of TES buffer. The solubilized proteins are separated into two equal fractions in order to have two cups containing the same quantity of proteins (1-1.5 mg/ml). One of the cups serves as a blank and the other to quantify the carbonyl groups. 500 μl of 12.5 mM DNPH is added to the measuring cup and 500 μl of 2M HCl to the blank cup. After incubation for 15 min at ambient temperature, the samples are precipitated with 500 μl of 30% TCA and centrifuged for 10 min at 15000 rpm. The pellet is taken up in 1 ml of 10% TCA and centrifuged again for 10 min at 15000 rpm. It is then washed 4 times in succession with ethanol ethyl acetate. The final pellet is heated for 30 min at 50° C. in a water bath. After another centrifugation of 5 min at 15000 rpm, the spectrophotometric reading is carried out on 800 μl of supernatant at 280 nm (protein assay) and at 380 nm (DNPH assay). The carbonyl content is expressed in nmol/mg of proteins.

III.2) Results:

The results obtained are shown in FIG. 1.

It is clear that malnutrition induces an increase in the carbonylation of proteins compared with the healthy rats (column 2 versus column 1). Renutrition also increases this phenomenon and the phenomenon of malnutrition-renutrition is accompanied by a marked increase in the carbonylation of muscle proteins (column 3 versus column 2). On the other hand, when the food is enriched with citrulline, this phenomenon disappears completely (column 4 versus column 3), the carbonylation of proteins being similar to that observed in the healthy rats.

### EXAMPLE 2

### Effect of Citrulline on Elderly Rats

Materials and Methods

I—Materials

All the chemical reagents used come from Sigma (Saint-Quentin-Fallavier, France). The L-citrulline was supplied free of charge by Laboratoires Biocodex (Compiègne, France).

II—Treatment of the Animals

Male Sprague-Dawley rats (Charles River Laboratoires, L′Arbresles, France) aged 25 months are placed in individual cages in a thermostatically-controlled atmosphere (23°±1° C.), and subjected to a 12-hour light-darkness cycle (darkness from 8 h to 20 h). The manager of the programme is authorized (no. 75.522) by the Ministry of Agriculture and Forestry to carry out this type of experiment. Moreover, the use and the treatment of the laboratory animals comply with legislation (D2001-486) and are in accordance with European legislation (Official Journal of the European Community L358 Dec. 18, 1986).

The rats are acclimatized for 2 weeks during which spontaneous food consumption is measured. They are fed with a standard diet (A04, UAR, Villemoisson-sur-Orge, France) containing 17% proteins, 3% lipids, 59% glucides and 21% water, fibres, vitamins and minerals. The average food intake during this period is 34.4 g/day.

III—Experimental Protocol

After the acclimatization period, the rats are randomized into 2 groups:

CIT group (n=5): the animals receive a standard diet enriched with citrulline (1 g/kg/d) for 1 month

AANE group (n=4): the animals receive a standard diet rendered isonitrogenous compared with that of the CIT group by the addition of non-essential amino acids (alanine, asparagine, glycine, serine, histidine, and proline in equimolar quantities) for 1 month.

At the end of the study, the animals were euthanized and their brains removed, weighed, frozen in liquid nitrogen and stored at −80° C. until the analyses were carried out.

Evaluation of the Carbonylation Level of the Proteins

The processes of oxidation of the proteins affecting the structure of the amino acids which comprise them are complex and all give rise to the formation of carbonyl groups. For this reason it was decided to carry out an overall evaluation of the oxidation of the muscle proteins by quantifying the carbonylated derivatives from a sample of 1 g of brain. The method used is therefore a non-specific amino-acid type technique based on the spectrophotometric assay of the complex formed by binding dinitrophenyl-hydrazine (DNPH) to the carbonyl groups. The cerebral proteins are isolated. The protein pellet is taken up in 600 μl of TES buffer. The solubilized proteins are separated into two equal fractions in order to have two cups containing the same quantity of proteins (1-1.5 mg/ml). One of the cups serves as a blank and the other to quantify the carbonyl groups. 500 μl of 12.5 mM DNPH is added to the measuring cup and 500 μl of 2M HCl to the blank cup. After incubation for 15 min at ambient temperature, the samples are precipitated with 500 μl of 30% TCA and centrifuged for 10 min at 15000 rpm. The pellet is taken up in 1 ml of 10% TCA and centrifuged again for 10 min at 15000 rpm. It is then washed 4 times in succession with ethanol ethyl acetate. The final pellet is heated for 30 min at 50° C. in a water bath. After another centrifugation of 5 min at 15000 rpm, the spectrophotometric reading is carried out on 800 μl of supernatant at 280 nm (protein assay) and at 380 nm (DNPH assay). The carbonyl content is expressed in μmol/g of organ.

IV—Results

| | Carbonylated proteins |
|---|---|
| | (μmol/g) |
| AANE | 180 ± 28 |
| CIT | 164 ± 12 |

Average ± ASD

The results obtained are shown in the table above. It is clear that a diet enriched with CIT in elderly healthy rats is accompanied by a decrease in the carbonylation of the cerebral proteins of the order of 10%.

1-9. (canceled)

10. Method for inhibiting an increase in the carbonylation of the relevant proteins observed in certain pathologies comprising the administration to a patient in need thereof of a composition comprising a therapeutically active amount of L-citrulline of the following formula (I)

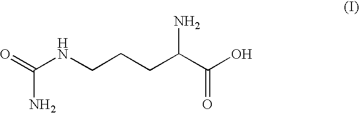

11. Method according to claim 10 wherein the pathology linked to an increase in the carbonylation of the relevant proteins is selected from the group comprising neurodegenerative diseases, retinopathy, rheumatoid polyarthritis, atherosclerosis, amyotrophic lateral sclerosis, cerebral ischemia, cataract problems, systemic infections (sepsis), pathologies associated with cutaneous aging and with senescence in tissues, pathologies associated with mitochondrial dysfunction, cachexia associated with undernutrition and with the increase in the longevity of mammals.

12. Method according to claim 10, characterized in that the composition is a pharmaceutical composition comprising L-citrulline or one of its pharmaceutically acceptable salts, a composition for cosmetic treatment comprising L-citrulline or one of its cosmetically acceptable salts, a food supplement comprising L-citrulline or a composition for nutraceutical treatment comprising L-citrulline or one of its nutraceutically acceptable salts.

13. Method according claim 10, characterized in that the L-citrulline is used such that the unit L-citrulline dose is about 2 g to about 20 g, in particular about 10 g, for a dosage of about 0.1 g/kg/day to about 0.5 g/kg/day, in particular about 0.25 g/kg/day at the rate of one to three daily doses, preferably in one dose.

14. Method according to claim 10, characterized in that the composition is in dried form, in the form of an aqueous solution, in hydroalcoholic or oily form, in the form of an oil-in-water or water-in-oil or multiple emulsion, an aqueous or oily gel.

15. Method according to claim 10, characterized in that the pharmaceutical composition is in a form administrable by oral, topical, enteral or parenteral route, the cosmetic composition in a form administrable by topical route, the nutraceutical composition and the food supplement in a form administrable by oral route.

16. Method according to claim 10, characterized in that in the case of a form administrable by enteral or parenteral route, it can be mixed with the enteral or parenteral nutrition or administered as a bolus in the enteral gastric probe or in a Y-tube in parenteral nutrition.

17. Method according to claim 10, characterized in that the composition also comprises one or more other compounds intended for the treatment of cachexia associated with undernutrition such as leucine, glutamine, arginine, ornithine and their various usable salts such as α-ketoglutarate or α-ketoisocaproate.

18. Method according to claim 10, characterized in that the composition is a pharmaceutica composition comprising L-citrulline and one or more other compounds intended for the treatment of cachexia associated with undernutrition such as leucine, glutamine, arginine, ornithine and their various usable salts such as α-ketoglutarate or α-ketoisocaproate, on their own or in a nutrient mixture intended for parenteral nutrition, or a mixture intended for enteral nutrition or a mixture intended for oral nutrition.

19. Composition comprising:
L-citrulline or a pharmaceutically or nutraceutically acceptable salt thereof,
and at least one other compound intended for the treatment of cachexia associated with undernutrition, such as leucine, glutamine, arginine, ornithine and their various usable salts such as α-ketoglutarate or α-ketoisocaproate, on their own or as a food mixture intended for parenteral nutrition, or as a mixture intended for enteral nutrition or a mixture intended for oral nutrition, as combination products for simultaneous, separate or sequential use, within the scope of the treatment of pathologies associated with an increase in the carbonylation of proteins.
